(19) United States
(12) Patent Application Publication
(10) Pub. No.: US 2017/0224557 A1
(43) Pub. Date: Aug. 10, 2017

(54) SaniHanz

(71) Applicant: Matilde Montalvo, Hoboken, NJ (US)

(72) Inventor: Matilde Montalvo, Hoboken, NJ (US)

(21) Appl. No.: 15/018,784

(22) Filed: Feb. 8, 2016

(51) Int. Cl. A61F 13/551; A41D 19/00

(52) U.S. Cl. CPC A61F 13/55185; A61F 13/5518; A41D 19/0075; A41D 19/002; A41D 2400/52

(57) ABSTRACT

A pair of disposable light biodegradable plastic/natural rubber or latex-free gloves into the production and packaging of tampons and feminine pads; alternately, the Sani-Hanz gloves could be produced with a tampon pocket in the center for use in holding a tampon of the lady's choice. With one glove to be used during the insertion process, and one in the removal process, the Sani-Hanz would not only act as a barrier to prevent the user's hand and clothing from contamination by blood spatter and blood-borne pathogens, but also act as a handy disposal bag for both the tampon applicator (first glove) and the soiled tampon (second glove).

### BACKGROUND OF THE INVENTION

1. Field of the Invention

The present invention relates generally to the field of sanitary aids for use in removing and inserting a tampon and more specifically relates to a pair of disposable biodegradable plastic or latex-free gloves into the production and packaging of tampons and/or feminine pads.

2. Description of the Related Art

A Tampon is a cylindrical mass of absorbent material, primarily used as a feminine hygiene product. At present, tampons are designed to be easily inserted into the vagina during menstruation and absorb the user's menstrual flow. A sanitary napkin, sanitary towel, sanitary pad, menstrual pad, maxi pad, or pad is an absorbent item worn by a woman or girl while she is menstruating, while she is recovering from vaginal surgery, for lochia (post birth bleeding), after an abortion, or in any other situation where it is necessary to absorb a flow of contaminated blood from her vagina. These pads are not to be confused with generally higher absorbency incontinence pads, which are worn by men or women who have urinary incontinence problems or experience stress incontinence. Menstrual pads may be used by some people for this purpose.

A few attempts have been made to solve problems found in sanitary aids for use in removing/inserting a tampon art. These are found in: U.S. Pat. No. 5,740,554 to B. Bernetiae Reed; U.S. Pub. No. 2006/0004338 to Torkildsen et al. and U.S. Pub. No. 6687911 to Martina Fitz.

None of the above inventions and/or patents, taken either singly or in combination, describe the invention as claimed. Thus, a need exists for a reliable, novel Sani-Hanz, a pair of disposable biodegradable plastic or latex-free gloves into the production and packaging of tampons and/or feminine pads to avoid the spread of contaminated blood spatter and blood-borne pathogens, which usually occurs during menstruation.

### BRIEF SUMMARY OF THE INVENTION

In view of the foregoing disadvantages inherent in the known sanitary aids, the present invention provides a novel Sani-Hanz. The general purpose of the present invention, which will be described subsequently in greater detail, is to provide a pair of biodegradable plastic or latex-free gloves into the production and packaging of tampons and/or feminine pads. Alternately, the Sani-Hanz gloves could be produced with a tampon pocket in the center for use in holding a tampon of the lady's choice. With one glove to be used during the insertion process, and one in the removal process, a novel Sani-Hanz would help reduce the existing environmental emission problems, while simultaneously avoiding the spread of contagious diseases. It would not only act as a barrier to prevent the user's hand and clothing from contamination by blood spatter and blood-borne pathogens, but also act as an the eco-friendly/compostable handy disposable bag for both the tampon applicator (first glove) and the soiled tampon (second glove). These advantages and unique features, methods of use and functionality, which are novel, are particularly pointed out and distinctly claimed in the concluding portion of the specification. Each unique aspect will become better understood with reference to the following drawings in detailed description.

### BRIEF DESCRIPTION OF THE DRAWINGS

The figures which accompany the written portion of this specification illustrate embodiments and method(s) of use for the present invention, Sani-Hanz, constructed and operative according to the teachings of the present invention.

FIG. 1 shows a perspective view illustrating a Sani-Hanz according to an embodiment of the present invention.

FIG. 2 shows a perspective step-by-step sketch illustration of a Sani-Hanz′ safe handling process for properly discarding both, the tampon applicator or the soiled tampon after insertion/removal.

The various embodiments of the present invention will hereinafter be described in conjunction with the appended drawings.

### DETAILED DESCRIPTION

As discussed above, embodiments of the present invention relate to a sanitary aids for use in removing and inserting a tampon device and more particularly to a Sani-Hanz, a pair of disposable biodegradable plastic or latex-free gloves into the production and packaging of tampons and/or feminine pads. Alternately, the Sani-Hanz gloves could be produced with a tampon pocket in the center for use in holding a tampon of the lady's choice. With one glove to be used during the insertion process, and one in the removal process, the Sani-Hanz would not only act as a barrier to prevent the user's hand and clothing from contamination by blood spatter and blood-borne pathogens, but also act as a handy disposal bag for both, the tampon applicator (first glove) and the soiled tampon (second glove).

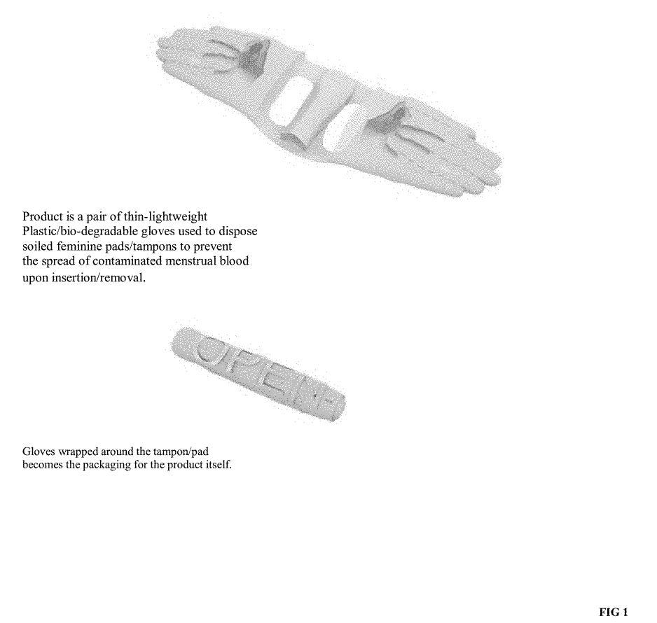

Referring now to the drawings in FIG. 1, the Sani-Hanz comprising a novel product offering consumers a practical solution to the aforementioned challenges and disadvantages. As the name implies, the Sani-Hanz would equip the individually packaged tampon (and perhaps also the feminine sanitary napkin) with a pair of lightweight, disposable, tear-off biodegradable plastic or latex-free gloves—one to be employed in the insertion of the tampon and disposal of the applicator, and one to be employed in the removal and disposal of the tampon. If produced within or as the tampon's packaging, the two gloves would be wrapped around the tampon and sealed, acting as its sanitary packaging.

Unwrapped or unrolled, one Sani-Hanz glove will be attached on one side of the tampon envelope, with a perforated tear-strip at the cuff or base of each glove. Again, alternately the Sani-Hanz gloves could be produced with a tampon/pad pocket between the two gloves. This pocket would serve to house almost any tampon or feminine pad the user desires. The light biodegradable plastic or latex-free gloves will be stretchable, and one-size fits all. The Sani-Hanz is cost-effective to produce in the embodiments, as shown in FIG. 1.

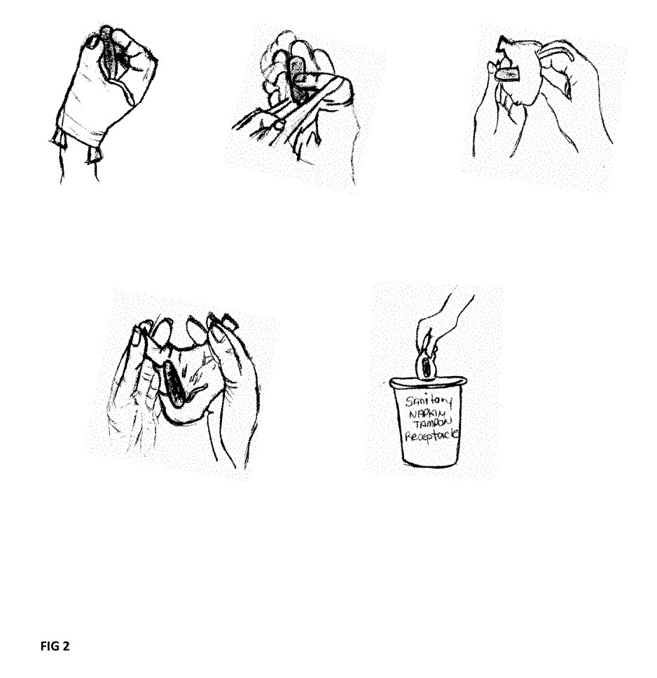

Instructions are as follows: Prior to insertion, before opening sealed tampon in the center, detach glove along perforated line. Wear the glove, then insert tampon as normally instructed. Referring now to the drawings in FIG. 2, after insertion, remove the glove while enveloping the disposable applicator into your gloved hand and wrapping it inside the glove. Now, you've turned the Sani-Hanz glove into a disposable baggie for proper discarding. Use small ties (provided with the Sani-Hanz) to properly seal the “baggie,” and discard the applicator and glove into a feminine hygiene receptacle, as shown in FIG. 2. Save and put away the other Sani-Hanz glove into your purse for tampon removal. The same process would be repeated with the second glove for later on discarding the soiled tampon.

The Sani-Hanz would be eco-friendly, simple to use, sleek in design and self-contained. Not only would the Sani-Hanz make the process of tampon insertion and removal fully hygienic and contamination-proof, but the gloves would also serve as convenient disposal bags for both the tampon applicator and the soiled tampon. Use of the Sani-Hanz would also prevent a woman from soiling her clothing through touch, again the unintentional consequence of handling a soiled tampon or napkin. Furthermore, the use of Sani-Hanz would also protect a woman from the inadvertent contamination of her hands with blood-borne pathogens. Thus, in order to protect the people with whom she comes into indirect contact, or subsequent contact through touch, a critical need exists for a Sani-Hanz. The Occupational, Safety and Health Administration. (OSHA) have set stringent regulations—found in Standard Numbers 1910.1030; 132 of the OSHA HealthCare and Infectious Diseases; OSHA Blood Borne Pathogens section—for doctors, nurses and healthcare workers, who handle contaminated blood with blood-borne pathogens during surgical procedures or emergency/life threatening situations for their safety and the safety others. Personal Protective Equipment (such as gloves) have been mandated to prevent the spread of infectious diseases such as (but not limited to), HBV and HIV. Consequently, the standards for handling soiled tampons/pads contaminated with blood-borne pathogens are no different. Clever in conception and thoughtful in design, the Sani-Hanz gloves would dramatically improve the use of feminine hygiene products in a healthful manner nationally or worldwide—and win the enthusiastic approval of the millions of girls, teens, and women who would use them.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claim:

1. A sanitary aids for use in removing and inserting a tampon device essentially as described herein.
